(19) United States
(12) Patent Application Publication
(10) Pub. No.: US 2025/0366822 A1
(43) Pub. Date: Dec. 4, 2025

(54) ULTRASONIC DIAGNOSTIC APPARATUS, STORAGE MEDIUM, AND ULTRASONIC DIAGNOSTIC METHOD

(71) Applicant: Konica Minolta, Inc., Tokyo (JP)

(72) Inventors: Tetsuya TANIGUCHI, Tokyo (JP); Sayuka ANDO, Tokyo (JP); Kazuya TAKAGI, Tokyo (JP)

(21) Appl. No.: 19/216,858

(22) Filed: May 23, 2025

(30) Foreign Application Priority Data
May 30, 2024 (JP) 2024-087780

(51) Int. Cl. A61B 8/00

(52) U.S. Cl. CPC A61B 8/42; A61B 8/5207

(57) ABSTRACT

An ultrasonic diagnostic apparatus that transmits an ultrasound wave into a subject, that receives the ultrasound wave reflected off a target tissue in the subject to obtain a reception signal, and that outputs an ultrasound image of the target tissue based on the reception signal, the ultrasonic diagnostic apparatus including: a hardware processor. The hardware processor, acquires a plurality of frames of the ultrasound image when an angle of an ultrasound probe is changed by a tilt-shift operation of the ultrasound probe, extracts a standard frame including the ultrasound image in a case where the ultrasound probe and the target tissue are orthogonal to each other from the plurality of acquired frames, and outputs the extracted standard frame.

### BACKGROUND OF THE INVENTION

### Technical Field

The present invention relates to an ultrasonic diagnostic apparatus, a storage medium, and an ultrasonic diagnostic method.

### Description of Related Art

In ultrasound examination of a motor organ and an anesthetized region, a fibrous tissue such as a muscle, a tendon, and a nerve bundle is imaged. When the fibrous tissue is imaged, a tilt-shift operation of changing an angle of an ultrasound probe is performed in a state where the ultrasound probe is pressed against a skin surface. In the tilt-shift operation, when the angle of the ultrasound probe with respect to the fibrous tissue is not appropriate, the tissue becomes an anisotropic reflection tissue from which a reflection signal cannot be obtained, and a high-brightness ultrasound image cannot be obtained.

As a technique for assisting scanning or the like of the ultrasound probe, there is a technique for acquiring information such as position and posture of the ultrasound probe using an accessory device and attaching the acquired information to the ultrasound image. As the accessory device, for example, a GPS module, a multi-viewpoint imaging camera (VR camera), or the like is used. Japanese Unexamined Patent Publication No. 2021-49211 describes an ultrasonic diagnostic apparatus that detects the position of the ultrasound probe in a three dimensional space using a position sensor.

However, in the conventional technique, the accessory device such as the position sensor is required in addition to the ultrasonic diagnostic apparatus, and there is a problem that the operation at the time of inspection is not simple and inexpensive. In addition, the conventional technique is a technique considering a depth direction which is a three dimensional space, and there is a problem in that the technique cannot be applied to the tilt-shift operation including a parallel movement of the ultrasound probe.

Therefore, in order to solve the above-described problem, an object of the present invention is to provide an ultrasonic diagnostic apparatus, a program in a storage medium, and an ultrasonic diagnostic method capable of acquiring an appropriate ultrasound image without using a special accessory device when a tilt-shift operation is performed with an ultrasound probe.

### SUMMARY OF THE INVENTION

According to an aspect of the present invention, an ultrasonic diagnostic apparatus reflecting one aspect of the present invention is the ultrasonic diagnostic apparatus that transmits an ultrasound wave into a subject, that receives the ultrasound wave reflected off a target tissue in the subject to obtain a reception signal, and that outputs an ultrasound image of the target tissue based on the reception signal, the ultrasonic diagnostic apparatus including:
a hardware processor,
wherein the hardware processor,
acquires a plurality of frames of the ultrasound image when an angle of an ultrasound probe is changed by a tilt-shift operation of the ultrasound probe,
extracts a standard frame including the ultrasound image in a case where the ultrasound probe and the target tissue are orthogonal to each other from the plurality of acquired frames, and
outputs the extracted standard frame.

According to another aspect of the present invention, an ultrasonic diagnostic apparatus reflecting one aspect of the present invention is the ultrasonic diagnostic apparatus that transmits an ultrasound wave into a subject, that receives the ultrasound wave reflected off a target tissue in the subject to obtain a reception signal, and that is capable of outputting an ultrasound image of the target tissue based on the reception signal, the ultrasonic diagnostic apparatus comprising:
a hardware processor,
wherein the hardware processor,
acquires a plurality of frames of the ultrasound image when an angle of an ultrasound probe is changed by a tilt-shift operation of the ultrasound probe, and
allocates angle information of the ultrasound probe with respect to a skin surface of a subject person when the tilt-shift operation is performed to each of the plurality of frames.

According to another aspect of the present invention, a non-transitory computer-readable storage medium reflecting one aspect of the present invention is the storage medium storing a program executed in a computer of an ultrasonic diagnostic apparatus that transmits an ultrasound wave into a subject, that receives the ultrasound wave reflected off a target tissue in the subject to obtain a reception signal, and that is capable of outputting an ultrasound image of the target tissue on the basis of the reception signal, the program allowing the computer to perform:
acquiring a plurality of frames of the ultrasound image when an angle of an ultrasound probe is changed by a tilt-shift operation of the ultrasound probe,
extracting a standard frame including the ultrasound image in a case where the ultrasound probe and the target tissue are orthogonal to each other from the plurality of acquired frames, and
outputting the extracted standard frame.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a block diagram of an ultrasonic diagnostic apparatus according to a first embodiment;

FIG. 2 is a flowchart showing an example of an operation of the ultrasonic diagnostic apparatus when performing an ultrasound examination by tilt-shift operation of the ultrasound probe according to the first embodiment;

FIG. 3A is a diagram illustrating a state of the ultrasound probe according to the first embodiment when the ultrasound probe is inclined at −0 deg;

FIG. 3B is a diagram illustrating an ultrasound image of a frame acquired by the ultrasound probe illustrated in FIG. 3A;

FIG. 4A is a diagram illustrating a state of the ultrasound probe when the ultrasound probe is inclined at +0 deg according to the first embodiment;

FIG. 4B is a diagram illustrating the ultrasound image of the frame acquired by the ultrasound probe illustrated in FIG. 4A;

FIG. 5 is a graph illustrating a change in average brightness of an image region in n frame(s) according to the first embodiment;

FIG. 6 is a diagram showing an ultrasound image of a frame acquired when the ultrasound probe according to the first embodiment is inclined at −0 deg;

FIG. 7 is a diagram showing the ultrasound image of the frame acquired when the ultrasound probe according to the first embodiment is inclined at +0 deg;

FIG. 8 is a graph illustrating the change in the average brightness in a plurality of image regions of each frame according to the first embodiment;

FIG. 9 is a diagram illustrating an example of the ultrasound image in a case where image information cut out from the image region of another frame is combined with the image region of a base frame according to the first embodiment;

FIG. 10A is an explanatory diagram for a case of estimating an angle of the ultrasound probe relative to a skin surface by using a first side face of a housing of the ultrasound probe according to a second embodiment;

FIG. 10B is an explanatory diagram for a case of estimating the angle of the ultrasound probe relative to the skin surface by using a second side surface of the housing of the ultrasound probe according to the second embodiment;

FIG. 11A is an explanatory diagram of a case where the angle of the ultrasound probe with respect to the skin surface is estimated by using a guide line indicated on a front face of the ultrasound probe according to the second embodiment;

FIG. 11B is an explanatory diagram of a case where the angle of the ultrasound probe with respect to the skin surface is estimated by using the guide line indicated on the front face of the ultrasound probe according to the second embodiment;

FIG. 12 is a flowchart showing an example of the operation of the ultrasonic diagnostic apparatus when allocating angle information and color tone information to each frame acquired by the tilt-shift operation of the ultrasound probe according to the second embodiment;

FIG. 13 is an explanatory diagram of a case where the prescribed angle value is equally divided by a number of frames n and the angle information and the color tone information are assigned to each frame according to the second embodiment;

FIG. 14 is a flowchart illustrating an example of the operation of the ultrasonic diagnostic apparatus when performing the ultrasound examination by tilt-shift operation of the ultrasound probe according to the second embodiment;

FIG. 15 is a flowchart illustrating an example of the operation of the ultrasonic diagnostic apparatus when performing the ultrasound examination by the tilt-shift operation of the ultrasound probe according to the second embodiment;

FIG. 16 is a diagram illustrating an example of an examination screen on which the angle information allocated to an extracted frame is displayed according to the second embodiment;

FIG. 17 is a diagram illustrating an example of the ultrasound image in a case where the image region or the like in the frame is subjected to color tone conversion according to the second embodiment;

FIG. 18 is a diagram illustrating an example of the examination screen on which the angle information and the like allocated to the frame according to the second embodiment are displayed;

FIG. 19 is a flowchart illustrating an example of the operation of the ultrasonic diagnostic apparatus when performing an ultrasound examination by the tilt-shift operation of the ultrasound probe according to the third embodiment;

FIG. 20 is a flowchart illustrating an example of operation of an ultrasonic diagnostic apparatus according to a third embodiment in a case where an ultrasound examination is performed by the tilt-shift operation of an ultrasound probe;

FIG. 21 is an explanatory diagram of the case of extracting a lumen region satisfying a threshold value condition from the ultrasound image of a predetermined frame according to the third embodiment;

FIG. 22 is a graph showing a tracking result when a lumen region extracted from n/2 frames is tracked between frames according to the third embodiment; and

FIG. 23 is a diagram illustrating an example of an examination screen on which the angle information allocated to an extracted frame is displayed according to the third embodiment.

### DETAILED DESCRIPTION

Hereinafter, one or more embodiments of the present invention will be described with reference to the drawings. However, the scope of the invention is not limited to the disclosed embodiments.

Below, with reference to the accompanying drawings, a detailed description will be given of an ultrasonic diagnostic apparatus, a program included in a storage medium, and an ultrasonic diagnostic method according to preferred embodiments of the present disclosure.

### EMBODIMENT

### Example Block Configuration of Ultrasonic Diagnostic Apparatus 1

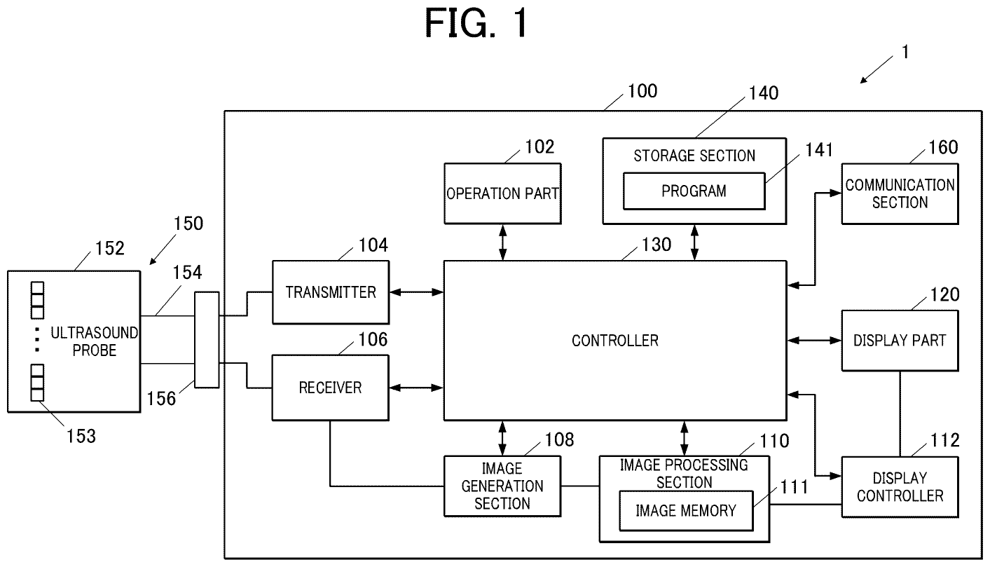

FIG. 1 is a block diagram of an ultrasonic diagnostic apparatus 1 according to a first embodiment. The ultrasonic diagnostic apparatus 1 is used by a user such as a doctor or a technician in a medical facility, a patient's home, or the like. As illustrated in FIG. 1, the ultrasonic diagnostic apparatus 1 includes an apparatus body 100 and an ultrasound probe 150 connected to the apparatus body 100. The apparatus body 100 is provided with an operation part 102 and a display part 120. In the apparatus body 100, a transmitter 104, a receiver 106, an image generation section 108, an image processing section 110, a display controller 112, a controller 130 (hardware processor), a storage section 140, and a communication section 160 are mounted.

The operation part 102 includes, for example, an operation panel including a plurality of buttons and a trackball, and a touch screen combined with the display part 120. The operation part 102 receives input instructions by various user operations, converts the received input instructions into electrical signals, and outputs the electrical signals to the controller 130.

The transmitter 104 supplies a driving signal, which is an electrical signal, to the ultrasound probe 150 under control of the controller 130. The transmitter 104 includes, for example, a clock generation circuit, a delay circuit, and a pulse generation circuit. The clock generation circuit generates a clock signal for determining transmission timing and transmission frequency of the driving signal. The delay circuit sets a delay time for each path provided in each probe 153 to be described later, and delays transmission of the driving signal by the set delay time. The delay circuit focuses a transmission beam constituted by an ultrasound wave. The pulse generation circuit generates a pulse signal as the driving signal at a predetermined cycle. The transmitter 104 drives, for example, a consecutive portion of a plurality of probes 153 to generate the ultrasound wave. The transmitter 104 performs scanning while shifting the probe 153 to be driven in the azimuth direction each time the ultrasound wave is generated.

The receiver 106 receives a reception signal, which is the electrical signal, from the ultrasound probe 150 under control of the controller 130. The receiver 106 includes, for example, an amplifier, an A/D conversion circuit, and a phasing addition circuit. The amplifier amplifies a reception signal at a preset amplification factor for each path provided in each probe 153. The A/D conversion circuit performs analog/digital conversion on the amplified reception signal. The phasing addition circuit gives the delay time to an A/D converted reception signal for each path provided in each probe 153 to adjust a time phase, and adds these. The phasing addition circuit generates sound ray data (sound ray signal) by phasing addition. Note that the receiver 106 may include an amplifier for amplifying the reception signal.

The image generation section 108 performs envelope detection processing, logarithmic compression, and the like on sound ray data supplied from the receiver 106. The image generation section 108 further adjusts at least one of a dynamic range and a gain for sound ray data and converts a brightness, to generate B-mode image data. The B-mode image data represents the intensity of a reception signal by brightness and is tomographic image information about a tissue of the subject. Image data generated by the image generation section 108 is not limited to the B-mode image data. Examples of the other scan modes (image modes) include an A-mode, an M-mode, and a 10 scan mode using the Doppler method. The Doppler method includes, for example, a color Doppler mode and a PWD. B-mode is an abbreviation for Brightness mode. A-mode is an abbreviation for Amplitude mode. M-mode is an abbreviation for Motion mode. PWD is an abbreviation for Pulsed Wave Doppler.

The image processing section 110 performs image processing on the B-mode image data output from the image generation section 108. The image processing section 110 performs image processing on the B-mode image data in accordance with various image parameters being set. The image processing section 110 includes an image memory 111 constituted by a semiconductor memory such as a DRAM. DRAM is an abbreviation for Dynamic Random Access Memory. The image processing section 110 stores the B-mode image data subjected to the image processing in the image memory 111 in units of frames under the control of the controller 130. Under the control of the controller 130, the image processing section 110 sequentially outputs the image data generated as described above to the display controller 112.

Under the control of the controller 130, the display controller 112 generates an image signal for display by performing coordinate conversion or the like on the received image data. The display controller 112 outputs the generated image signal for display to the display part 120.

The display part 120 is, for example, a display device such as a liquid crystal display or an organic EL. EL is an abbreviation for Electro Luminescence. The display part 120 displays the ultrasound image of a tissue, an organ, or the like of a subject based on the image signal for display output from the display controller 112 on the screen according to the control of the controller 130. The ultrasound image may be a still image or a moving image.

The controller 130 includes a processor such as a CPU and a memory such as a RAM. CPU is an abbreviation for Central Processing Unit. RAM is an abbreviation for Random Access Memory. The CPU reads various programs 141 stored in the storage section 140, develops the programs in the RAM, and executes various kinds of processing related to an ultrasound examination in cooperation with the programs 141. The CPU may be constituted by a single processor or a plurality of processors.

In the present embodiment, the controller 130, which is a computer included in the ultrasonic diagnostic apparatus 1, functions as at least an acquisition section, an extraction section, and an output section. The processor such as the CPU of the controller 130 executes the program 141 stored in the storage section 140 or the like to realize various functions such as an acquisition section, an extraction section, and an output section. The acquisition section acquires a plurality of frames of the ultrasound image when the ultrasound probe 150 is changed in a range of a predetermined angle by a tilt-shift operation of the ultrasound probe 150. The tilt-shift operation refers to an operation of rotating and inclining of the ultrasound probe 150 within a predetermined angle range in a state in which the ultrasound probe 150 is pressed against a skin surface of the subject. The extraction section extracts, from the plurality of frames acquired by the acquisition section, a frame (standard frame) including the ultrasound image in a case where the ultrasound probe 150 and a target tissue are orthogonal to each other. The target tissue includes a fibrous tissue such as a muscle, a tendon, and a nerve bundle. The term orthogonal means that a central axis of a slice beam of the ultrasound probe 150 is orthogonal or substantially orthogonal to the fibrous tissue when the fibrous tissue as the target tissue is observed in a short axis direction. When a direction in which the fibrous tissue extends is defined as a long axis direction, a short axis direction is a direction orthogonal to the long axis direction. In addition, the target tissue includes not only the muscle, the tendon, and the nerve bundle, which are so-called collections of fibrous tissues, but also a luminal tissue such as a blood vessel. The interior of a lumen of a luminal structure is a tissue such as blood that is rendered echoless or hypoechoic, and is not an anisotropically reflective tissue. However, a wall tissue such as a blood vessel wall exhibits a characteristic of anisotropic reflection although not so much as the muscle or the like, and good visualization can be obtained at the time of orthogonalization similarly to the muscle or the like. The output section outputs ultrasound image data of the frame extracted by the extraction section to the display part 120.

The storage section 140 includes at least one storage module, for example, an HDD, an SSD, a ROM, and a RAM. HDD is an abbreviation of Hard Disk Drive. SSD is an abbreviation for Solid State Drive. ROM is an abbreviation of Read Only Memory. RAM is an abbreviation for Random Access Memory. The storage section 140 stores, for example, a system program, an application program, and various types of data received by the communication section 160. For example, the storage section 140 stores a program 141 for executing processing related to the ultrasound examination, processing for outputting ultrasound image data and the like, and the like.

The communication section 160 includes, for example, an NIC, a LAN adapter, and a communication module including a receiver and a transmitter. NIC is an abbreviation for Network Interface Card. The communication section 160 communicates various kinds of data, information, and the like with an external device such as a medical image management system via a network, for example.

The ultrasound probe 150 includes a head part 152, a cable 154, and a connector 156. The head part 152 is a portion to be pressed against a body surface of a subject person. The head part 152 is provided with a plurality of probes 153 formed of piezoelectric elements. The probe 153 transmits the ultrasound wave to the subject on the basis of a driving signal transmitted from the apparatus body 100, and receives a reflected wave reflected by a target tissue in the subject. For example, the plurality of probes 153 may be arranged in a scanning direction in a one dimensional array, or may be arranged in a two dimensional array (matrix). The number of probes 153 can be arbitrarily set. As a scanning method of the ultrasound probe 150, a linear scanning method, a convex scanning method, a sector scanning method, or the like can be adopted.

The cable 154 has one end electrically connected to the head part 152 and the other end electrically connected to the connector 156. The connector 156 is connected to the apparatus body 100. Note that the communication between the apparatus body 100 and the ultrasound probe 150 is not limited to wired communication using the cable 154. The communication method between the apparatus body 100 and the ultrasound probe 150 may be wireless communication using UWB or the like. UWB is an abbreviation for Ultra Wide Band.

### Operation Example of Ultrasonic Diagnostic Apparatus 1

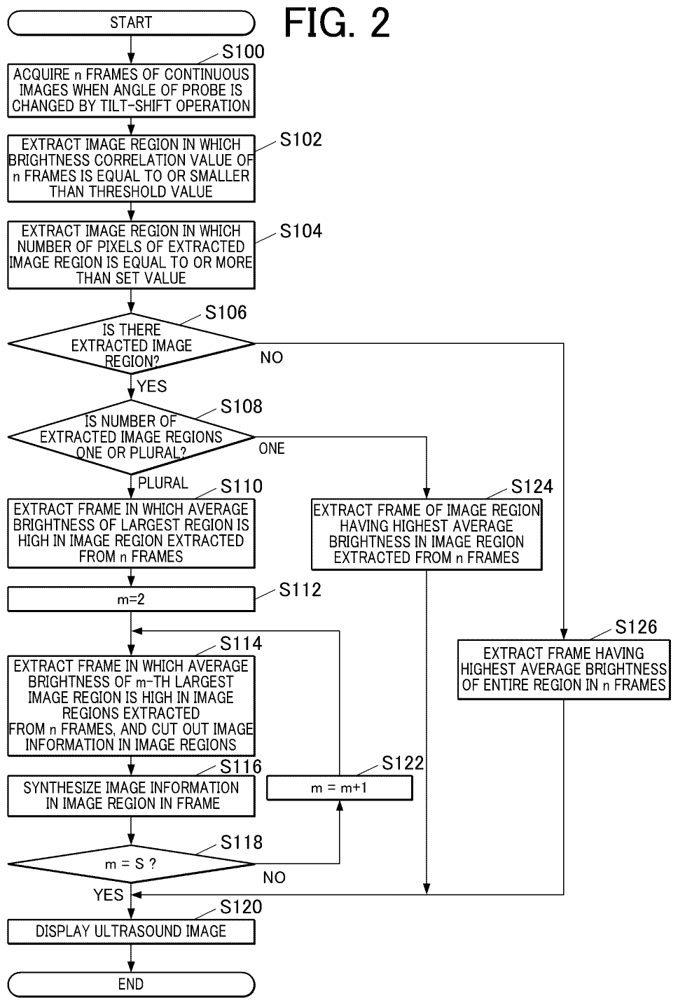

FIG. 2 is a flowchart showing an example of an operation of the ultrasonic diagnostic apparatus 1 when performing the ultrasound examination by the tilt-shift operation of the ultrasound probe 150 according to the first embodiment. The controller 130 realizes each processing of an acquiring step, an extracting step, an outputting step, and the like described below by executing the program 141 and the like of the storage section 140.

A user presses the ultrasound probe 150 against the skin surface of the subject and causes the ultrasound probe 150 to transmit the ultrasound wave to the target tissue during the tilt-shift operation of the ultrasound probe 150. The receiver 106 sequentially receives the reflection signals reflected by the target tissue in a case where the angle of the ultrasound probe 150 is changed by the tilt-shift operation. The controller 130 continuously acquires n frames of the ultrasound image based on the reflection signals received by the receiver 106 (step S100). N is a positive integer.

The controller 130 extracts an image region in which an inter-frame brightness correlated value is equal to or less than the threshold value CV in the image region in each of the acquired n frames of the ultrasound images (step S102). The brightness correlation value is a value indicating a brightness change between the image regions located at the same or substantially the same position in each of the n frames of the ultrasound images acquired by the tilt-shift operation. The threshold value CV is the threshold value for extracting the image region having a low brightness correlated value between image regions located at the same or substantially the same position in n frames of the ultrasound images. Here, as a method of specifying the image region from the entire image, the following methods are exemplified. For example, there is a method in which the image is divided into m×n (m and n are arbitrary integers) regions, the brightness correlation value is calculated for each of the regions, and the region having the brightness correlation value equal to or smaller than the threshold value is extracted. The number of divisions is the largest in a case where the calculation is performed in pixel units of the image, but in this case, the amount of calculation is large. Therefore, an appropriate number of divisions is set in consideration of the observation target and a computing capacity of the device. In addition, the shape of the divided image region may not be rectangular, and each divided image region may partially overlap with the adjacent divided image region. For example, the divided image region is set in a circular shape, and the adjacent divided image region is set by shifting their positions so as to partially superimpose each other. This method makes it possible to cover the entire image region even with a circular divided image region shape. The threshold value may be a predetermined fixed value, but a method is preferable in which the threshold value is adaptively set from a result of the obtained brightness correlation value. The threshold value is set to, for example, “extract the lowest 20% of the brightness correlation values”.

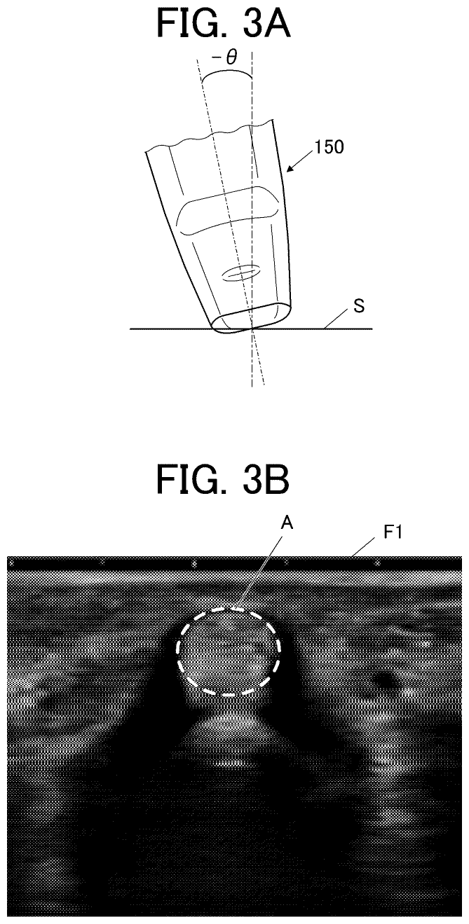

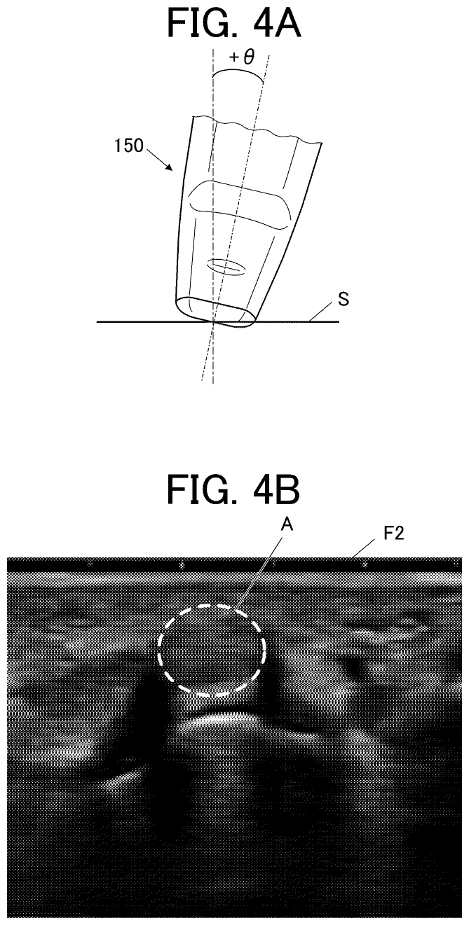

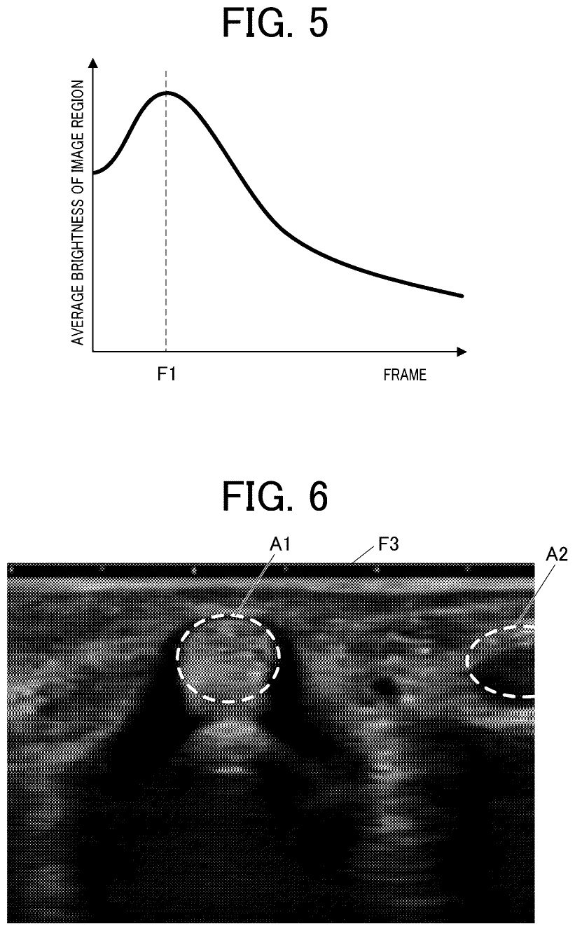

FIG. 3A is a diagram illustrating a state of the ultrasound probe 150 when the ultrasound probe 150 is inclined at −0 deg according to the first embodiment. FIG. 3B is a diagram illustrating the ultrasound image of a frame F1 acquired by the ultrasound probe 150 illustrated in FIG. 3A. FIG. 4A is a diagram illustrating the state of the ultrasound probe 150 when the ultrasound probe 150 is inclined at +0 deg according to the first embodiment. FIG. 4B is a diagram illustrating the ultrasound image of a frame F2 acquired by the ultrasound probe 150 illustrated in FIG. 4A. Note that the ultrasound image is an image including a flexor tendon of a middle finger and the like. FIG. 5 is a graph illustrating a change in average brightness of an image region in n frame(s) according to the first embodiment. In FIG. 5, the horizontal axis represents a number of frames, and the vertical axis represents the average brightness of the image region.

In the ultrasound images of the frames F1 and F2, the region including, for example, the flexor tendon of the middle finger is defined as an image region A. The image region A of the frame F2 is a region in which the ultrasound waves from the ultrasound probe 150 are not orthogonal to a short axis direction of the tendon (fibrous tissue) and which anisotropically reflects the ultrasound waves from the ultrasound probe 150. Therefore, the image region A in the frame F2 has lower brightness than the image region A in the frame F1. As a result, the brightness change between the image regions A in the frame F1 and the frame F2 becomes large, and the brightness-correlation value decreases correspondingly. The brightness correlation value between the image regions A in the frame F1 and the frame F2 is less than or equal to the threshold value CV. In this case, the controller 130 extracts the image region A as the image region in which the brightness correlation value is equal to or less than the threshold value CV in the frame F1 and the frame F2.

The controller 130 extracts the image region in which the number of pixels of the image region extracted from each ultrasound image of n frames is equal to or larger than a set value TP (step S104). The set value TP is the threshold value for extracting the region which is not a pixel unit influenced by noise or the like but the region which is cohesive as the tissue. Therefore, the controller 130 removes the image region having a small number of pixels among the extracted image regions.

The controller 130 determines whether the image region is extracted from each of the n frames of the ultrasound images (step S106). That is, the controller 130 determines whether or not there is the image region in which the brightness correlation value is equal to or less than the threshold value CV between the n frames.

When determining that the image region is extracted from each of the n frames of the ultrasound images, the controller 130 proceeds to step S108. The controller 130 determines whether the number of image regions extracted from each of the n frames is one (singular) or plural (step S108). When determining that the number of image regions extracted from each of the ultrasound images of the n frames is one, the controller 130 proceeds to step S124.

The controller 130 extracts the frame including the image region having the highest average brightness in the image regions extracted from the n frames of the ultrasound images (step S124). For example, as illustrated in FIGS. 3B and 5, when the image region A in the frame F1 has the highest average brightness, the controller 130 extracts the frame F1 including the image region A having the highest average brightness from among the n frames. Upon extracting the frame including the image region having the high average brightness, the controller 130 proceeds to step S120.

The controller 130 allows the screen of the display part 120 to display the ultrasound image of the frame having the highest average brightness of the entire region among the extracted n frames (step S120). To be specific, the controller 130 outputs the ultrasound image data of the frame F1 illustrated in the FIG. 3B to the display part 120, and causes the display part 120 to display the ultrasound image of the frame F1 on the examination screen.

On the other hand, when determining that a plurality of image regions have been extracted from the n frames of the ultrasound images, the controller 130 proceeds to step S110. The controller 130 extracts an image region having the largest number of pixels among the plurality of extracted image regions. Subsequently, the controller 130 extracts the frame including the image region having the highest average brightness among the plurality of extracted image regions from the n frames (step S110).

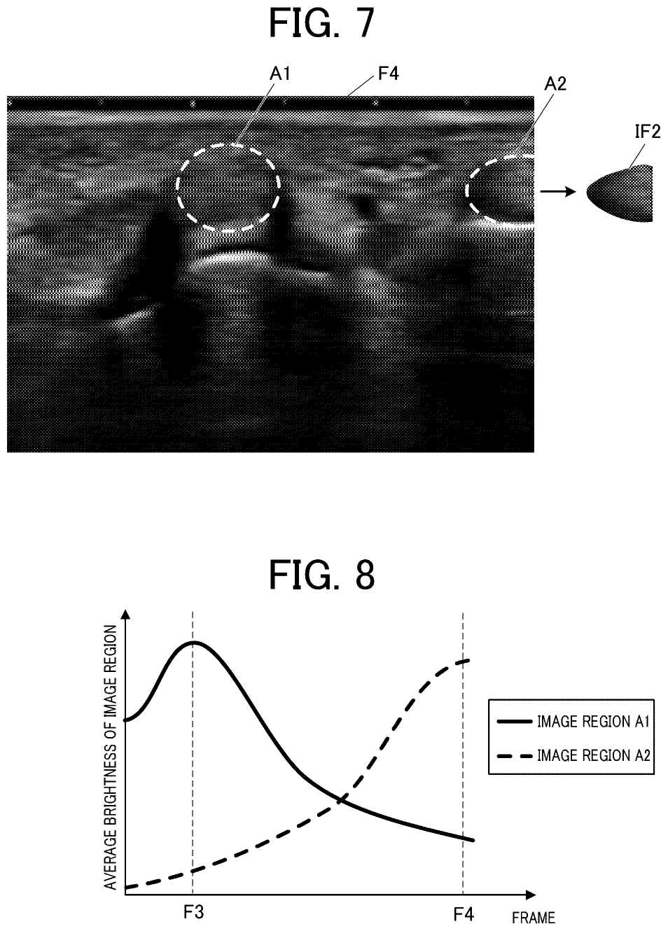

FIG. 6 is a diagram showing the ultrasound image of a frame F3 acquired when the ultrasound probe 150 according to the first embodiment is inclined at −0 deg. FIG. 7 is a diagram showing the ultrasound image of a frame F4 acquired when the ultrasound probe 150 according to the first embodiment is inclined at +0 deg. FIG. 8 is a graph illustrating the change in the average brightness in the image regions A1 and A2 of each frame according to the first embodiment. In FIG. 8, the horizontal axis represents the number of frames, and the vertical axis represents the average brightness of the image region. Note that in the first embodiment, an example in which two frames are used will be described for convenience, but actually, a large number of frames are included.

Each of the frame F3 and the frame F4 includes a plurality of image regions Am. For example, the first image region Am is the flexor tendon of the middle finger, and the second image region Am is the flexor tendon of an index finger. In the present embodiment, an identification number is assigned in accordance with the number of pixels (area) of each image region Am. Specifically, when there are the image region of the flexor tendon of the middle finger and the image region of the flexor tendon of the index finger as the image region Am, as shown in FIGS. 6 and 7, the image region of the flexor tendon of the middle finger is larger than the image region of the flexor tendon of the index finger. Therefore, in the frame F3 and the frame F4, the controller 130 allocates m=1 to the image region Am of the flexor tendon of the middle finger, and allocates m=2 to the image region Am of the flexor tendon of the index finger.

First, the controller 130 performs processing related to the frame including the image region A1 of m=1. The controller 130 extracts the frame in which the average brightness of the image region A1 is the highest among the frame F3 and the frame F4. As illustrated in FIGS. 6, 7, and 8, the average brightness of the image region A1 of the frame F3 is higher than the average brightness of the image region A1 of the frame F4. Therefore, the controller 130 extracts the frame F3 as a base frame from among the frame F4 and the frame F2. The base frame is the frame to be displayed on the examination screen of the display part 120. Although the selection and extraction of the base frame are performed using the average brightness here, the selection and extraction may be performed using an image brightness histogram. This is performed by using a skewness which is a feature amount indicating a deviation of symmetry from a normal distribution of the brightness histogram, a kurtosis which is the feature amount indicating sharpness (sharpness and width of a bottom of a distribution) with respect to the normal distribution, or the like. The skewness is obtained by the following Expression (1). The kurtosis is obtained by the following Expression (2).

$$\begin{matrix}[ Expression 1 ] & \\ \frac{n}{( n - 1 ) ( n - 2 )} {{\sum }_{i = 1}}^{n} {( \frac{{x}_{i} - \overline{x}}{s} )}^{3} & ( 1 )\end{matrix}$$

$$\begin{matrix}[ Expression 2 ] & \\ \frac{n ( n + 1 )}{( n - 1 ) ( n - 2 ) ( n - 3 )} {{\sum }_{i = 1}}^{n} \frac{{( {x}_{i} - \overline{x} )}^{4}}{{s}^{4}} - \frac{3 {( n - 1 )}^{2}}{( n - 2 ) ( n - 3 )} & ( 2 )\end{matrix}$$

n: sample size, x̅: average value of each data xᵢ (i:, 1, 2, . . . , n), s: standard deviation

Next, when the processing related to the image region A1 of m=1 is completed, the controller 130 increments a variable m and sets m=2 (step S112).

The controller 130 extracts a frame in which the average brightness of the m-th largest image region Am is the highest among the image regions extracted from the n frames of the ultrasound images. Subsequently, the controller 130 cuts out the image information IFm in the image region Am of the extracted frame (step S114). Hereinafter, since m=2 is set in step S112, processing related to the frame including the image region A2 will be described.

To be specific, the controller 130 extracts the frame having the highest average brightness among the image regions A2 of the frame F3 and the frame F4. As illustrated in FIGS. 6, 7, and 8, the average brightness of the image region A2 of the frame F4 is higher than the average brightness of the image region A2 of the frame F3. Therefore, the controller 130 extracts the frame F4 from among the frame F3 and the frame F4. Subsequently, as shown in FIG. 7, the controller 130 cuts out the image information IF2 in the image region A2 of the extracted frame F4. When the image information is cut out, brightness information is stored in the system memory or the like together with position information based on the position information of the image region A2. At this time, region position information of the image region A2 may be used as it is, but in a case where the target tissue is a tendon, a method may be adopted in which an edge between a low-brightness portion and a high-brightness portion in the image region A2 is detected, and the image information is cut out with the edge as a boundary. In this case, the image information IF2 includes the flexor tendon of the index finger.

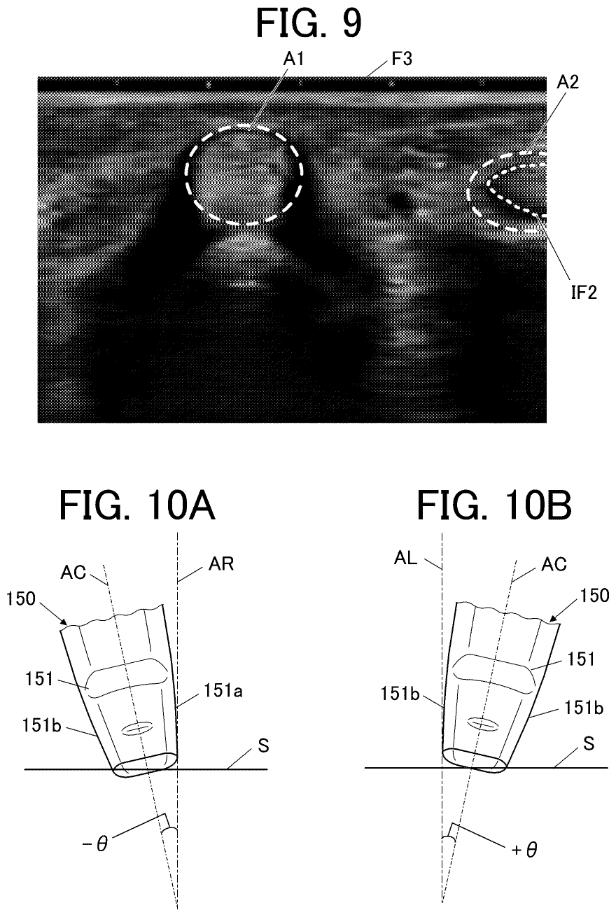

The controller 130 synthesizes the image information IFm cut out from another frame with the m-th largest image region Am in the ultrasound image of the frame set as the base frame (step S116). Synthesis is performed by replacing (overwriting) the brightness information at the corresponding position based on the brightness information and the position information of the image information IFm temporarily stored in the system memory or the like. At this time, in order to make the boundary between the base frame and the image region inconspicuous, boundary blurring processing may be performed such that the pixels corresponding to the boundary have an average brightness of the base frame and the image information IFm. FIG. 9 is a diagram showing an example of the ultrasound image in a case where image information IF2 cut out from the image region A2 of the frame F4 is synthesized with the image region A2 of the frame F3 according to the first embodiment. As illustrated in FIG. 9, the controller 130 pastes the cut out image information IF2 in the image region A2 of the frame F4 to the image region A2 of the frame F3 that is the base frame. Thus, the image region A2 can be expressed with high brightness in the ultrasound image of the frame F3.

The controller 130 determines whether m=s has been established (step S118). For example, s is the number of image regions rendered in one frame. When m=s has been established, the controller 130 proceeds to step S120. On the other hand, when determining that m=s has not been established, the controller 130 proceeds to step S122. The controller 130 increments m (m=m+1) and returns to step S114.

The controller 130 causes the display part 120 to display, on its screen, the ultrasound image of the frame set as the base frame (step S120). To be specific, as illustrated in FIG. 9, the controller 130 outputs the ultrasound image data of the frame F3 to the display part 120, and causes the display part 120 to display the ultrasound image data of the frame F3 on the examination screen. The ultrasound image of the frame F3 includes the image region A1 including the flexor tendon of the middle finger and the image region A2 including the flexor tendon of the index finger. The image region A2 is the image information IF2 cut out from the image region A2 of the frame F4 different from the frame F3. The image region A1 and the image region A2 are each displayed with high brightness.

Returning to step S106, when the controller 130 determines that the image region is not extracted in the n frames of the ultrasound image, the process proceeds to step S126. That is, this is the case where the brightness change between image regions in the n frames is small and the brightness correlation value is high. In this case, all the ultrasound images of the acquired n frames have brightness equal to or higher than a certain level, and are appropriate ultrasound images.

The controller 130 extracts the frame having the highest average brightness of the entire region among the n frames (step S126). Specifically, the controller 130 calculates the average brightness of all the pixels in each frame, and extracts the frame having the highest average brightness among them. After extracting the frame having high average brightness, the controller 130 proceeds to step S120.

The controller 130 allows the screen of the display part 120 to display the ultrasound image of the frame having the highest average brightness of the entire region among the extracted n frames (step S120).

According to the first embodiment, the following effectiveness can be exhibited. In a case where the fibrous tissue such as the muscle, the tendon, or the nerve bundle is observed in the short axis direction by the tilt-shift operation, if the ultrasound probe 150 is not orthogonal to the fibrous tissue, the reflection signal may not be appropriately received due to the anisotropic reflection by the fibrous tissue. Therefore, the accurate tilt-shift operation of causing the ultrasound probe 150 to be orthogonal to the fibrous tissue is required. However, there is a problem that it is difficult for the operator with low proficiency to make the ultrasound probe 150 accurately orthogonal to the fibrous tissue in the tilt-shift operation.

According to the first embodiment, when the correlation brightness value between the image regions including the fibrous tissue by the plurality of frames is low, the controller 130 can extract the frame including the image region having the highest average brightness from the plurality of frames. That is, by performing the tilt-shift operation, the frame in a case where the ultrasound probe 150 and the image region are most orthogonal to each other can be automatically generated. Thus, an optimal ultrasound image can be acquired while performing the tilt-shift operation of the ultrasound probe 150 without using the accessory device. As a result, the optimal ultrasound image can be acquired by a simple operation without performing a special operation, and an increase in cost can also be prevented because no accessory device is required. In addition, the operator can acquire the optimum ultrasound image only by performing the tilt-shift operation of the ultrasound probe 150 without being conscious of the anisotropic reflection of the fibrous tissue.

Furthermore, when each frame includes image regions including a plurality of fibrous tissues, the optimum angle with respect to the ultrasound probe 150 may be different for each of the plurality of image regions. In this case, the frame including the optimum image region is different for each of the plurality of image regions. Therefore, there is a problem that the plurality of image regions cannot be observed and compared at the same time in the ultrasound image of one frame displayed on the examination screen.

In contrast, according to the first embodiment, the following processing is performed in a case where each frame includes at least the first image region and the second image region. The controller 130 extracts, as the base frame, the frame including the first image region having the highest average brightness among the first image regions. Next, the controller 130 extracts the frame including the second image region having the highest average brightness among the second image regions, and cuts out the image information from the second image region of the frame. Next, the controller 130 synthesizes the cut out image information with the second image region of the base frame. Thus, in addition to the high-brightness first image region, the combined high-brightness second image region can be displayed in the ultrasound image of one frame that is the base frame. As a result, in one examination screen, a plurality of image regions displayed with high brightness can be simultaneously observed and compared.

### Second Embodiment

In the second embodiment, the angle information of the ultrasound probe 150 at the time of acquisition of each frame is allocated to each frame acquired by the tilt-shift operation of the ultrasound probe 150. Hereinafter, the differences from the first embodiment will be mainly described, constituent elements substantially common to the first embodiment will be assigned with the same reference signs, and common description will be omitted or simplified.

### [Specified Value Angle in Tilt-Shift Operation of Ultrasound Probe 150]

A prescribed angle value for specifying and estimating an inclinable angle of the ultrasound probe 150 with respect to the skin surface S during the tilt-shift operation is set for the ultrasound probe 150. The prescribed angle value is prescribed by using a housing shape of the ultrasound probe 150 and guide information written on the housing of the ultrasound probe 150. In the present embodiment, a means for defining the angle of the ultrasound probe 150 by using the housing shape of the ultrasound probe 150 is referred to as a first angle estimation means. A means for defining the angle of the ultrasound probe 150 using the guide information described on the ultrasound probe 150 is referred to as a second angle estimation means.

First, the first angle estimation means will be described. FIG. 10A is an explanatory diagram in a case of estimating the angle of the ultrasound probe 150 with respect to the skin surface S by using a first side surface 151a of the housing 151 of the ultrasound probe 150. FIG. 10B is an explanatory diagram for a case of estimating the angle of the ultrasound probe 150 with respect to the skin surface S by using a second side surface 151b of the housing 151 of the ultrasound probe 150. The housing 151 of the ultrasound probe 150 is provided with the first side surface 151a and the second side surface 151b opposite thereto. The first side surface 151a and the second side surface 151b of the housing 151 are configured by inclined surfaces which are inclined at a predetermined angle with respect to a central axis AC of the ultrasound probe 150. In the ultrasound probe 150, an axis passing through a first side surface 151a of the ultrasound probe 150 is defined as a reference axis AR, and an axis passing through a second side surface 151b of the ultrasound probe 150 is defined as a reference axis AL.

As illustrated in FIG. 10A, in a case where the first side surface 151a is orthogonal to the skin surface S, the angle formed by the central axis AC of the ultrasound probe 150 and the reference axis AR is-0 deg. As illustrated in FIG. 10B, when the second side surface 151b is orthogonal to the skin surface S, the angle formed by the central axis AC of the ultrasound probe 150 and the reference axis AL is +0 deg. Here, since the ultrasound probe 150 has a bilaterally symmetrical shape in a front view, −0 and +0 are the same angle range. The user can operate the ultrasound probe 150 within a range of the prescribed angle value by tilting the ultrasound probe 150 such that the first side surface 151a and the second side surface 151b of the ultrasound probe 150 are orthogonal to the skin surface S.

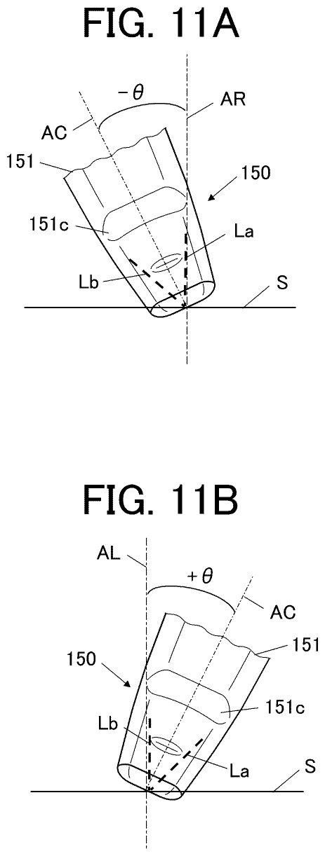

Next, the second angle estimating means will be described. FIG. 11A is an explanatory diagram in a case where the angle of the ultrasound probe 150 with respect to the skin surface S is estimated using a guide line La indicated on a front 151c of the ultrasound probe 150. FIG. 11B is an explanatory diagram in a case where the angle of the ultrasound probe 150 with respect to the skin surface S is estimated using a guide line Lb indicated on the front 151c of the ultrasound probe 150. As the guide information of the second angle estimation unit, for example, two guide lines La and Lb indicated on the front 151c of the ultrasound probe 150 can be used.

V-shaped guide lines La and Lb are indicated on the front 151c of the ultrasound probe 150. The guide lines La and Lb are arranged symmetrically with respect to the central axis AC of the ultrasound probe 150 as a reference. An acute-angled portion of the V shape is positioned at the center of a leading end of the ultrasound probe 150. In the present embodiment, the angle formed by the guide line La and the guide line Lb is the prescribed angle value. Specifically, when the guide line La is orthogonal to the skin surface S, the angle formed by the central axis AC of the ultrasound probe 150 and the guide line La is-0 deg. When the guide line Lb is orthogonal to the skin surface S, the angle formed by the central axis AC of the ultrasound probe 150 and the guide line Lb is +0 deg. Since the guide lines La and Lb are symmetrical with respect to the central axis AC of the ultrasound probe 150, −0 and +0 are in the same angular range. The user can operate the ultrasound probe 150 within a range of the prescribed angle value by tilting the ultrasound probe 150 such that the guide line La and the guide line Lb of the ultrasound probe 150 are orthogonal to the skin surface S.

### Example of Operation of Ultrasonic Diagnostic Apparatus 1 in Case of Allocating Angle Information and Color Tone Information to Each Frame

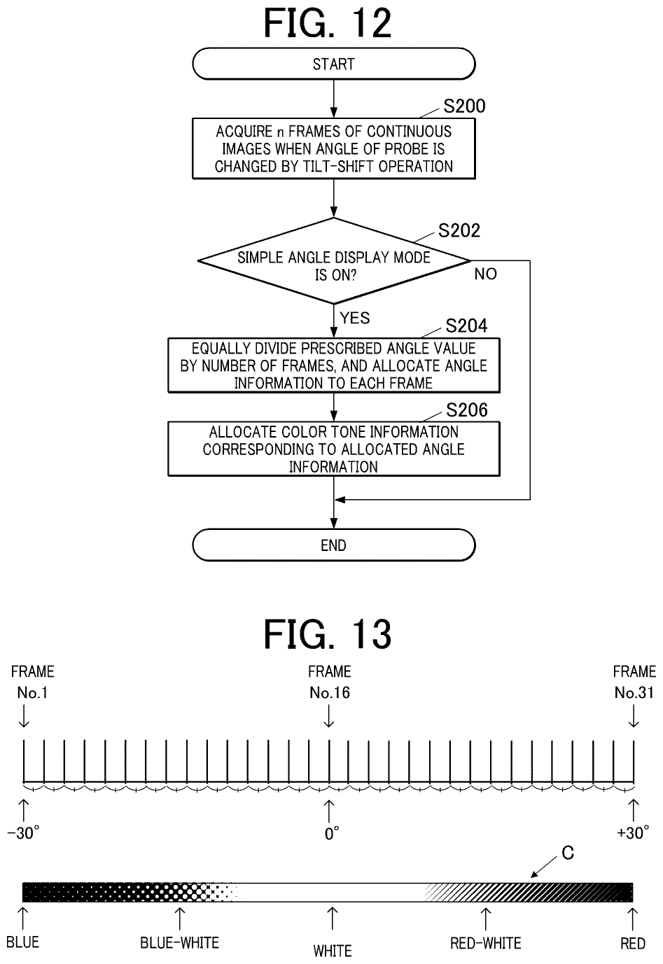

FIG. 12 is a flowchart showing an example of the operation of the ultrasonic diagnostic apparatus 1 when allocating the angle information and the color tone information to each frame acquired by the tilt-shift operation of the ultrasound probe 150 according to the second embodiment. The controller 130 executes the program 141 and the like in the storage section 140, to thereby implement each of processes such as an acquisition step and a setting step described below.

The controller 130 acquires each ultrasound image of n frames in a case where the ultrasound probe 150 is changed within the range of the specified angle with respect to the skin surface S by the tilt-shift operation (step S200). As a means for supporting the operation of tilting the ultrasound probe 150 to the prescribed angle value, the above-described first angle estimation means may be used, or the second angle estimation means may be used.

For example, when the first angle estimation means is used, the user inclines the ultrasound probe 150 with respect to the skin surface S so that the first side surface 151a of the ultrasound probe 150 is orthogonal to the skin surface S as illustrated in FIG. 10A. Thus, the ultrasound probe 150 can be inclined by −0 deg that is the prescribed angle value with respect to the skin surface S. From this position, as illustrated in FIG. 10B, the user inclines the ultrasound probe 150 in a direction opposite to the skin surface S so that the second side surface 151b of the ultrasound probe 150 is orthogonal to the skin surface S. Thus, the ultrasound probe 150 can be inclined by +0 deg that is the prescribed angle value with respect to the skin surface S. In this way, it is possible to perform the tilt-shift operation of the ultrasound probe 150 within a range of a preset angle.

The controller 130 determines whether a simple angle display mode is set to ON (step S202). The simple angle display mode is a mode in which, when the ultrasound image of the predetermined frame is displayed on the display part 120, the angle information of the ultrasound probe 150 at the time of acquiring the predetermined frame is additionally displayed. Turning on and off of the simple angle display mode may be appropriately set by operation of an icon, a button, or the like displayed on the examination screen or the like. When determining that the simple angle display mode is set to ON, the controller 130 proceeds to step S204. On the other hand, when determining that the setting of the simple angle display mode is OFF, the controller 130 ends the series of processing without performing processing related to the simple angle display mode.

The controller 130 equally divides the preset prescribed angle value by the number n of the acquired frames, and assigns the angle information to each frame (step S204). The controller 130 functions as a setting section. FIG. 13 is an explanatory diagram of a case where the prescribed angle value is equally divided by the number of frames n and the angle information and the color tone information are assigned to each frame according to the second embodiment. Hereinafter, a case where the prescribed angle value is 60° and the number of frames is 31 will be described. In this case, the user performs the tilt-shift operation of the ultrasound probe 150 in the range of −30° (−0) to +30° (+0) with respect to the perpendicular to the skin surface S. It is assumed that a speed of the tilt-shift operation is constant. With this tilt-shift operation, the controller 130 acquires 31 frames of continuous ultrasound images. Subsequently, the controller 130 equally divides the prescribed angle value by the number of frames n using the following Expression (3), and calculates the movement angle per frame at the time of the tilt-shift operation of the ultrasound probe 150.

movement angle per frame=0(deg)×2/(n−1) (3)

The controller 130 substitutes the preset prescribed angle value of 60° and the acquired number of frames of 31 into Expression (1). Thus, the controller 130 obtains 2° as the angle of movement or change per frame. Next, the controller 130 allocates the angle information to each frame based on the calculated moving angle per frame at the time of the tilt-shift operation of the ultrasound probe 150. For example, as illustrated in FIG. 13, the controller 130 allocates −30° as the angle information to the first frame, and allocates −16° as the angle information to the eighth frame. The controller 130 allocates 0° as the angle information to the 16-th frame, which is the middle of the consecutive images. The controller 130 allocates 16° as the angle information to the 24-th frame and allocates 30° as the angle information to the 31-st frame. In this way, the controller 130 allocates unique angle information to all other frames.

The controller 130 allocates, to each frame, the color tone information corresponding to the angle information allocated to each frame (step S206). As shown in FIG. 13, the color tone information is defined by a color bar C which is changed continuously or stepwise, for example, as “blue to blue-white to white to red-white to red”. In the present embodiment, blue-type is indicated by dot hatching, red-type is indicated by oblique hatching, and white is indicated by white hatching. The dot size increases from blue to blue-white, and the width of the diagonal line decreases from red to red-white. Each frame acquired by the tilt-shift operation is allocated a unique color from among the color bars C. The controller 130 may use a lookup table in which the angle information and the color tone information are associated with each other to allocate the color tone information to each frame. For example, as illustrated in FIG. 13, the controller 130 allocates blue as the color tone information to the 1-st frame, and allocates blue-white as the color tone information to the 8-th frame. Similarly, the controller 130 allocates white as the color tone information to the 16-th frame, allocates red-white as the color tone information to the 24-th frame, and allocates red as the color tone information to the 31-st frame. In this way, the controller 130 allocates the unique color tone information to other frames. After the allocation of the color tone information is completed, the controller 130 creates a lookup table in which the frame number, the angle information, and the color tone information are associated with each other. The created lookup table can be saved in a storage section such as the storage section 140.

### Example of Operation of Ultrasonic Diagnostic Apparatus 1 During Ultrasound Examination

Next, a method of acquiring the ultrasound image in a case where the above-described simple angle display mode is combined with the method of extracting the frame including the image region having the high average brightness from the plurality of frames described in the first embodiment will be described.

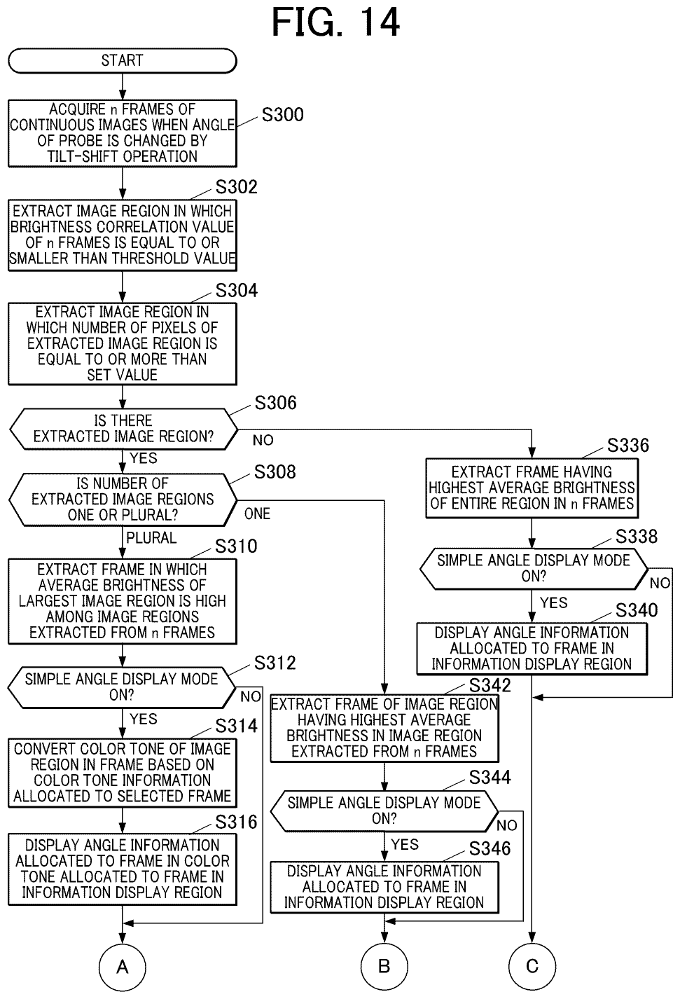

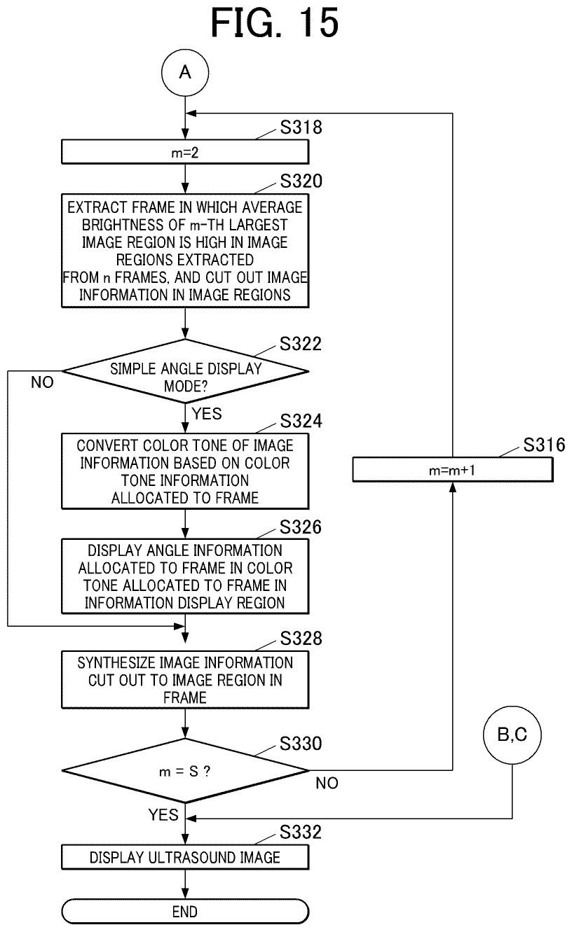

FIG. 14 and FIG. 15 are flowcharts illustrating an example of the operation of the ultrasonic diagnostic apparatus 1 when performing the ultrasound examination by the tilt-shift operation of the ultrasound probe 150 according to the second embodiment. The controller 130 realizes each processing of the acquiring step, the extracting step, the outputting step, and the like described below by executing the program 141 and the like of the storage section 140.

As illustrated in FIG. 14, the controller 130 continuously acquires n frames of the ultrasound images in a case where the ultrasound probe 150 is changed within a range of the specified angle with respect to the skin surface S by the tilt-shift operation (step S300). The controller 130 extracts the image region in which the brightness correlated value between frames is equal to or less than the threshold value CV in the image region in each of the acquired n frames of the ultrasound images (step S302). The controller 130 extracts the image region in which the number of pixels of the image region extracted from n frames is equal to or larger than the set value TP (step S304).

The controller 130 determines whether the image region is extracted in each of the n frames of the ultrasound images (step S306). When determining that the image region is extracted in each of the n frames of the ultrasound images, the controller 130 proceeds to step S308. The controller 130 determines whether the number of image regions extracted from each of the n frames is one or plural (step S308).

When determining that the number of image regions extracted from each of the ultrasound images of the n frames is one, the controller 130 proceeds to step S342. On the other hand, when determining that a plurality of image regions have been extracted from the n frames of the ultrasound images, the controller 130 proceeds to step S310.

First, a case in which one image region is extracted from each of the ultrasound images of n frames will be described. The controller 130 extracts the frame including the image region having the highest average brightness from the image regions extracted from the respective n frames (step S342). For example, as illustrated in FIG. 3B, when the image region A in the frame F1 has the highest average brightness, the controller 130 extracts the frame F1 including the image region A having the highest average brightness from among the n frames. Upon extracting the frame including the image region A having the high average brightness, the controller 130 proceeds to step S344.

The controller 130 determines whether the simple angle display mode is set to ON (step S344). When determining that the simple angle display mode is set to ON, the controller 130 proceeds to step S346. On the other hand, when determining that the setting of the simple angle display mode is OFF, the controller 130 proceeds to step S332.

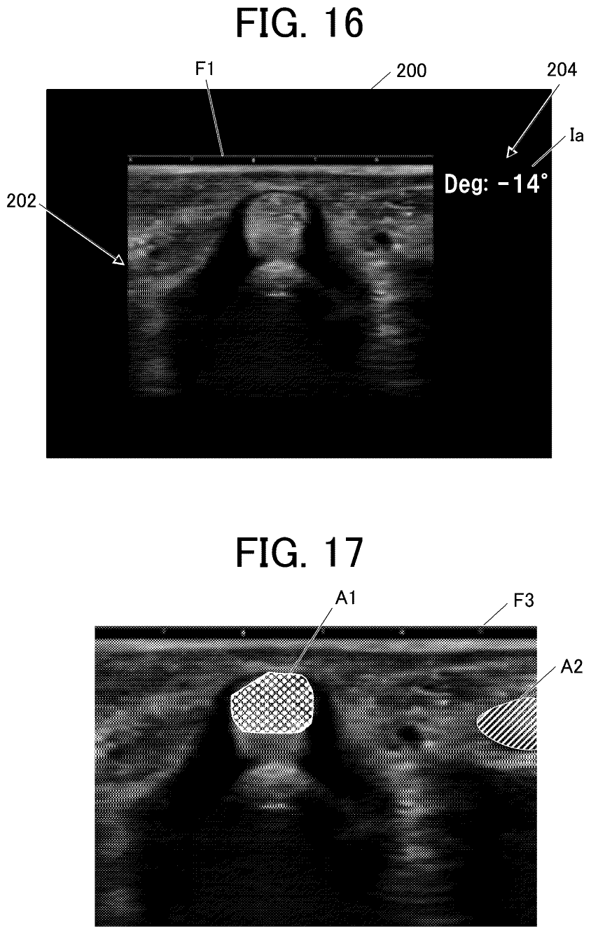

The controller 130 displays the angle information allocated to the frame extracted in step S342 in an information display region provided on the examination screen of the display part 120 (step S346). FIG. 16 is a diagram illustrating an example of an examination screen 200 on which the angle information Ia allocated to the extracted frame F1 is displayed. An image display region 202 in which the ultrasound image of the frame F1 is displayed is provided substantially in the center of the examination screen 200. On the right side of the examination screen 200, an information display region 204 is provided which displays the angle information Ia of the ultrasound probe 150 when the frame F1 displayed in the image display region 202 is acquired. In the information display region 204, “deg −14°” that is the angle information Ia allocated to the frame F1 is displayed. In FIG. 16, the ultrasound image is displayed in the image display region 202 for convenience of description, but the ultrasound image is not actually displayed in the image display region 202 at this timing.

After displaying the angle information Ia on the examination screen 200, the controller 130 proceeds to step S332. The controller 130 allows the examination screen 200 of the display part 120 to display the ultrasound image of the frame including the image region having the highest average brightness (step S332). To be specific, as illustrated in FIG. 16, the controller 130 displays the ultrasound image of the frame F1 including the image region A having the highest average brightness in the image display region 202 of the examination screen 200. Thus, the ultrasound image of the frame F1 including the image region A having the highest average brightness and the angle information Ia of the ultrasound probe 150 at the time of acquiring the ultrasound image are simultaneously displayed on the examination screen 200.

Next, a case in which the plurality of image regions are extracted from the n frames of the ultrasound image will be described. The controller 130 extracts the image region having the largest number of pixels among the plurality of extracted image regions. Subsequently, the controller 130 extracts the frame having the highest average brightness in the extracted image region from the n frames (step S310). An example of a case where there are the plurality of image regions will be described with reference to FIGS. 6 and 7 described above. To be specific, when a plurality of image regions Am are included in the frame F3 and the frame F4, respectively, the controller 130 extracts the image region A1 as the image region having the largest number of pixels. Subsequently, the controller 130 extracts the frame F3 as the frame in which the average brightness of the image region A1 is highest.

The controller 130 determines whether the simple angle display mode is set to ON (step S312). When determining that the simple angle display mode is set to ON, the controller 130 proceeds to step S314. On the other hand, when determining that the setting of the simple angle display mode is OFF, the controller 130 proceeds to step S318.

The controller 130 performs color tone conversion on the image region in the frame based on the color tone information allocated to the frame extracted in step S310 (step S314). First, the controller 130 performs processing related to the frame including the image region A1 of m=1. FIG. 17 is a diagram illustrating an example of the ultrasound image in a case where the image region A1 or the like in the frame F3 is subjected to color tone conversion according to the second embodiment. Note that the frame F3 is the 8-th frame of the 31 frames. In this case, as shown in FIG. 13, “blue-white” is allocated as the color tone information of the frame F3. The controller 130 refers to the lookup table and acquires the color tone information of “blue-white” allocated to the 8-th frame F3.

The controller 130 converts the image region A1 in the ultrasound image of the 8-th frame F3 into the color tone of “blue-white” based on the acquired color tone information.

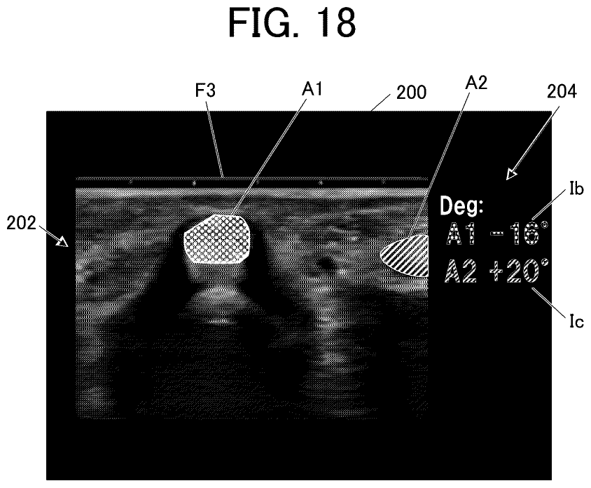

The controller 130 performs color tone conversion on the angle information allocated to the frame based on the color tone information allocated to the frame and displays the angle information in the information display region 204 of the examination screen 200 (step S316). When the extracted frame F3 is the 8-th frame, as illustrated in FIG. 13, “−16°” is allocated as angle information Ib of the frame F3. FIG. 18 is a diagram illustrating an example of the examination screen 200 on which the angle information Ib and the like allocated to the frame F3 according to the second embodiment are displayed. In the information display region 204, “deg −16°” that is the angle information Ib allocated to the frame F3 is displayed. The angle information Ib is displayed in “blue white” which is the color tone information allocated to the frame F3. Note that although the angle information Ic, the ultrasound image, and the like are displayed in FIG. 18 for convenience, the angle information Ic and the like are not displayed at this stage.

As shown in FIG. 15, when the processing regarding the image region A1 of m=1 is completed, the controller 130 increments the variable m to set m=2 (step S318).

The controller 130 extracts the frame in which the average brightness of the m-th largest image region is the highest among the plurality of image regions extracted from the n frames. Subsequently, the controller 130 cuts out the image information IFm in the image region of the extracted frame (step S320). Hereinafter, since m=2 is set in step S318, processing related to the frame including the image region A2 will be described.

To be specific, the controller 130 extracts the frame having the highest average brightness among the image regions A2 of the frame F3 and the frame F4. As illustrated in FIGS. 6 and 7, the average brightness of the image region A2 of the frame F4 is higher than the average brightness of the image region A2 of the frame F3. Therefore, the controller 130 extracts the frame F4 from among the frame F3 and the frame F4. Subsequently, the controller 130 cuts out the image information IF2 in the image region A2 of the extracted frame F4.

The controller 130 determines whether the simple angle display mode is set to ON (step S322). When determining that the simple angle display mode is set to ON, the controller 130 proceeds to step S324. On the other hand, when determining that the setting of the simple angle display mode is OFF, the controller 130 proceeds to step S328.

The controller 130 performs the color tone conversion on the image information IFm based on the color tone information allocated to the frame of the image information IFm cut out in step S320 (step S324). Note that the order of the frame F4 extracted in step S320 is the 26-th frame among the 31 frames. In this case, as illustrated in FIG. 13, “red-white” is allocated as the color tone information of the frame F4. The controller 130 refers to the lookup table and acquires the color tone information of “red-white” allocated to the 26-th frame F4. The controller 130 converts the image information IF2 cut out from the 26-th frame F4 into the color tone of “red-white” on the basis of the acquired color tone information.

The controller 130 performs the color tone conversion on the angle information allocated to the frame based on the color tone information allocated to the frame and displays the angle information in the information display region 204 of the examination screen 200 (step S326). When the frame F4 extracted in step S320 is the 26-th frame, “+20°” is allocated as the angle information Ic of the frame F4 as shown in FIG. 13. In the information display region 204, “deg +20°” that is the angle information Ic allocated to the frame F4 is displayed below the angle information Ib of the frame F3. The angle information Ic is displayed in “red-white” which is the color tone information allocated to the frame F4.

The controller 130 synthesizes the image information IFm cut out in another frame with the m-th largest image region in the frames as the base frame (step S328). To be more specific, as shown in FIG. 17, the controller 130 pastes and synthesizes the image information IF2 cut out from the image region A2 of the frame F4 to the image region A2 of the frame F3 which is the base frame. Thus, in the ultrasound image of the frame F3, the image region A2 can also be expressed with high brightness in addition to the image region A1.

The controller 130 determines whether m=s has been established (step S330). For example, s is the number of image regions rendered in one frame. When the variable m has become s, the controller 130 proceeds to step S332. On the other hand, when determining that m=s has not been established, the controller 130 proceeds to step S316. The controller 130 increments m (m=m+1) and returns to step S318.

The controller 130 causes the display part 120 to display, on the examination screen 200, the ultrasound image of the frame set as the base frame (step S332). As illustrated in FIG. 18, the controller 130 displays the ultrasound image of the frame F3 set as the base frame on the examination screen 200 of the display part 120. The image region A1 of the ultrasound image is displayed in “blue-white”. The image information IF2 obtained by cutting out the inside of the image region A2 of the frame F4 is synthesized with the image region A2 of the ultrasound image. The synthesized image information IF2 is displayed in “red-white”. Thus, the image region A1 and the image region A2 with high brightness can be displayed on the examination screen 200 of the display part 120. That is, images of the flexor tendon of the middle finger and the flexor tendon of the index finger can be drawn with high brightness on the examination screen 200 of the display part 120.

On the other hand, returning to step S306 shown in FIG. 14, when the controller 130 determines that the image region is not extracted in the n frames of the ultrasound image, the process proceeds to step S336. That is, this is the case where the brightness change between the image regions in the n frames is small and the brightness correlation value is high. In this case, all the ultrasound images of the acquired n frames have brightness equal to or higher than a certain level, and are appropriate ultrasound images.

The controller 130 extracts the frame having the highest average brightness of the entire region among the n frames (step S336). After extracting the frame having high average brightness, the controller 130 proceeds to step S338.

The controller 130 determines whether the simple angle display mode is set to ON (step S338). When determining that the simple angle display mode is set to ON, the controller 130 proceeds to step S340. On the other hand, when determining that the setting of the simple angle display mode is OFF, the controller 130 proceeds to step S332.

The controller 130 displays the angle information allocated to the frame extracted in step S336 in the information display region 204 of the examination screen 200 (step S340). After causing the angle information to be displayed, the controller 130 proceeds to step S332. The controller 130 causes the image display region 202 of the examination screen 200 to display the ultrasound image of the frame extracted in step S336 (step S332). On the examination screen 200, the ultrasound image of the frame including the high-brightness image region and the angle information of the ultrasound probe 150 when the ultrasound image is acquired are simultaneously displayed.

According to the second embodiment, the same effects as those of the first embodiment described above can be achieved. Furthermore, according to the second embodiment, for a predetermined frame acquired by the tilt-shift operation, the angle information of the ultrasound probe 150 with respect to the skin surface S when the frame is acquired is displayed on the examination screen 200. Therefore, it is possible to relatively or semi-quantitatively grasp information regarding the depth direction of the ultrasound probe 150 on the two dimensional examination screen 200. Further, even when there are a plurality of image regions as the target tissue, since the angle information of each image region is displayed on the examination screen 200, the relative positional relationship of each image region can also be grasped. Thus, the angle information of each image region can be used not only as new diagnostic information but also as index information for rehabilitation.

### Third Embodiment

In the third embodiment, the frame including the high-brightness lumen region is extracted based on the depth of the lumen region in the ultrasound image. Hereinafter, a method of acquiring the ultrasound image in a case where the simple angle display mode described in the second embodiment is combined with the method of extracting the frame including the high-brightness lumen region based on the depth of the lumen region in the ultrasound image according to the third embodiment will be described. Furthermore, constituent elements that are substantially common to the first embodiment and the second embodiment have the same reference numerals, and the common description is omitted or simplified.

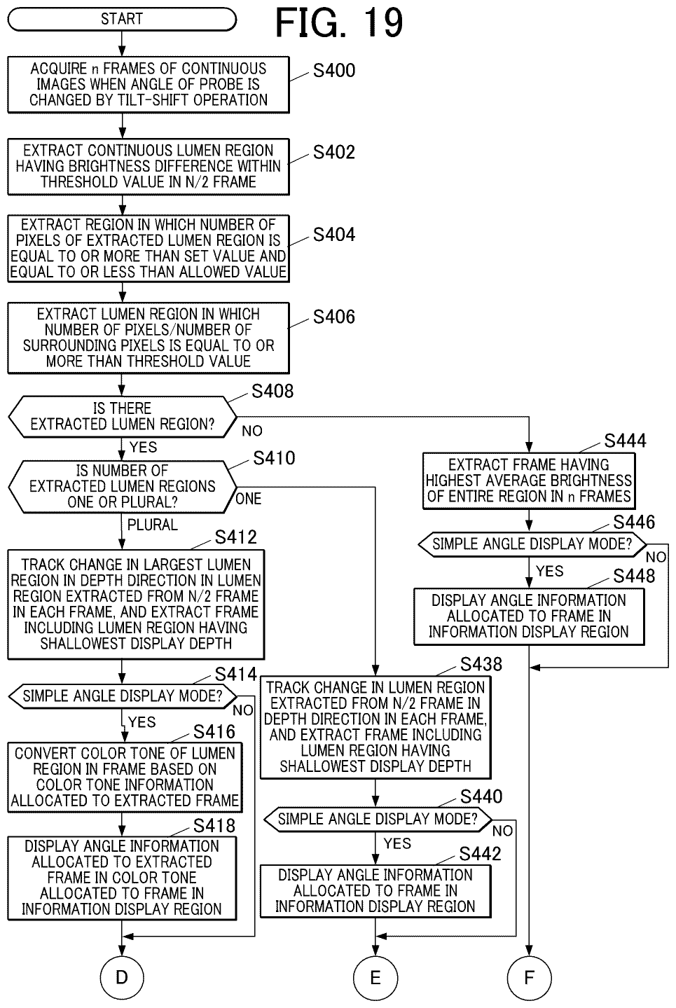

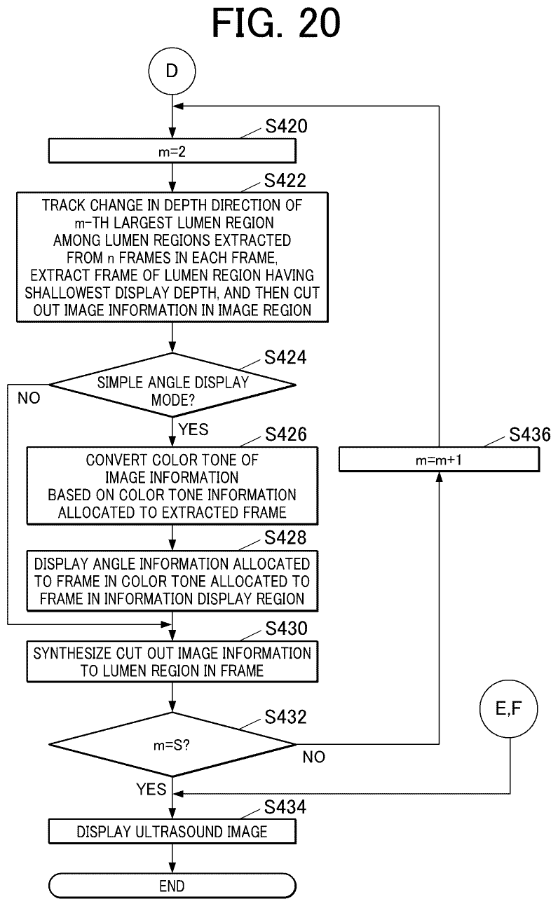

FIG. 19 and FIG. 20 are flowcharts illustrating an example of the operation of the ultrasonic diagnostic apparatus 1 when performing the ultrasound examination by the tilt-shift operation of the ultrasound probe 150 according to the third embodiment. The controller 130 realizes each processing of an acquiring step, an extracting step, an outputting step, and the like described below by executing the program 141 and the like of the storage section 140.

As illustrated in FIG. 19, the controller 130 continuously acquires n frames of the ultrasound images in a case where the ultrasound probe 150 is changed within a range of the specified angle with respect to the skin surface S by the tilt-shift operation (step S400).

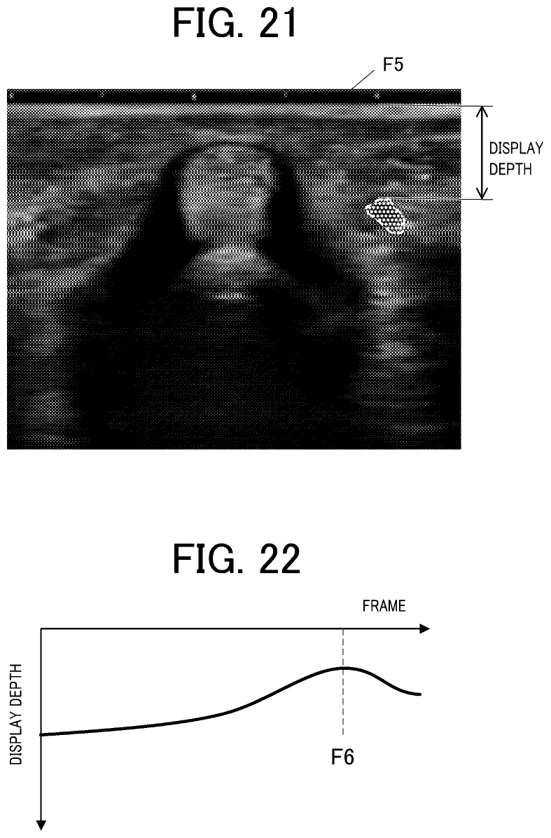

The controller 130 extracts the lumen region in which the brightness difference is equal to or less than the threshold value TB in the frame corresponding to the (n/2)-th frame (rounded up) of the n frames (step S402). Here, the frame in which the tilt-shift angle has a value closest to 0° with respect to the skin surface S is selected as the base frame. Unlike the muscle or the like, the inside of a lumen structure does not have a high echo even at the time of orthogonality. Therefore, it is difficult to select the base frame based on average brightness, but the feature amount of the brightness histogram such as the skewness or the kurtosis may be used. The threshold value TB is the maximum brightness difference that allows adjacent pixels to be a similar region, and is a threshold value for extracting the lumen region from the ultrasound image. FIG. 21 is an explanatory diagram of the case of extracting a lumen region B satisfying a condition of a threshold value TB from the ultrasound image of the predetermined frame according to the third embodiment. For example, when the number of acquired frames is 31, the controller 130 extracts the 16-th frame F5. The controller 130 extracts consecutive lumen regions B having a brightness difference equal to or smaller than the threshold value TB from the extracted ultrasound image of the 16-th frame. Furthermore, in order to selectively extract the lumen region whose inside is in a state of no echo to low echo, it is preferable to set an upper limit value of the brightness in addition to the brightness difference and perform extraction.

The controller 130 extracts the region in which the number of pixels of the lumen region extracted from the n frames is equal to or larger than the set value TP and equal to or smaller than an allowable value MP (step S404). The set value TP is the threshold value for extracting the region which is not the pixel unit influenced by noise or the like but the region which is cohesive as the tissue. The allowable value MP is the maximum number of pixels to be extracted as the lumen region, and is the threshold value for excluding a non-signal region in a deep portion. The set value TP and the allowable value MP may be specified values that change in association with the display image size, or may allow the user to select any numeric value on the screen or the like.

The controller 130 extracts the lumen region where the number of pixels of the lumen region extracted from the n frame/the number of surrounding pixels is equal to or more than the threshold value TC (step S406). The threshold value TC is a threshold value for excluding the region having a large ratio of the number of surrounding pixels to the number of pixels of an image, such as a linear or an irregular shape, to extract the lumen region with high accuracy.

The controller 130 determines whether the lumen region is extracted in each of the n frames of ultrasound images (step S408). When determining that the lumen region is extracted in the ultrasound images of the n frames, the controller 130 proceeds to step S410.

The controller 130 determines whether the number of lumen regions extracted from each of the n frames is one or plural (step S410). When determining that the number of lumen regions extracted from each of the ultrasound images of the n frames is one, the controller 130 proceeds to step S438. On the other hand, when determining that the plurality of lumen regions are extracted from the n frames of the ultrasound image, the controller 130 proceeds to step S412.

First, a case where the number of lumen regions extracted from each of the ultrasound images of the n frames is one will be described. The controller 130 tracks a change in the depth direction of the lumen region extracted from the n/2 frame in each frame. Subsequently, the controller 130 extracts, from among the tracked lumen regions in the respective frames, the frame including the lumen region having the shallowest display depth (step S438). This utilizes the fact that, for example, when the tilt-shift operation is performed in a state where the contact point of the ultrasound probe 150 is fixed to the skin surface S, the distance is the shortest when the contact point is orthogonal to the lumen region, and the contact point is displayed at a shallow depth. FIG. 22 is a graph illustrating a tracking result when the lumen region B extracted from n/2 frames is tracked between frames according to the third embodiment. In FIG. 22, the horizontal axis represents the frame and the vertical axis represents the display depth. Note that the display depth is based on the shallowest part of the lumen region of each frame as a standard. The controller 130 refers to the graph and extracts a frame F6 as the frame having the shallowest depth in the lumen region B of the n frames.

The controller 130 determines whether the simple angle display mode is set to ON (step S440). When determining that the simple angle display mode is set to ON, the controller 130 proceeds to step S442. On the other hand, when determining that the setting of the simple angle display mode is OFF, the controller 130 proceeds to step S434.

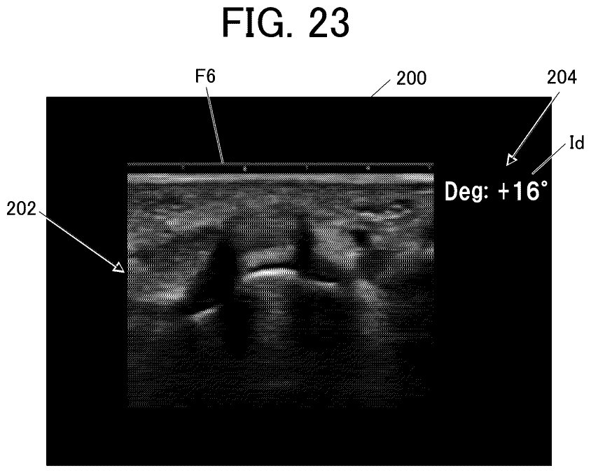

The controller 130 displays the angle information Id allocated to the frame extracted in step S438 in the information display region 204 of the examination screen 200 (step S442). FIG. 23 is a diagram showing an example of the examination screen 200 on which the angle information Id allocated to the extracted frame F6 according to the third embodiment is displayed. In the information display region 204, for example, “deg +16°” which is the angle information Id allocated to the frame F6 is displayed. The angle information Id is the angle of the ultrasound probe 150 with respect to the skin surface S when the frame F6 is acquired by the tilt-shift operation. In FIG. 23, the ultrasound image is displayed in the image display region 202 for convenience of description, but the ultrasound image is not actually displayed in the image display region 202 at this timing.

The controller 130 causes the image display region 202 of the examination screen 200 to display the ultrasound image of the frame extracted in step S438 (step S434). To be specific, as illustrated in FIG. 23, the controller 130 displays the ultrasound image of the frame F6 including the lumen region B having the shallowest depth in the image display region 202 of the examination screen 200. Accordingly, the ultrasound image of the frame F6 including the lumen region B having the highest brightness and the angle information Id of the ultrasound probe 150 when the ultrasound image is acquired are simultaneously displayed on the examination screen 200.

Subsequently, a case in which a plurality of lumen regions is extracted from each of n frames of ultrasound images will be described. The controller 130 tracks a change in the depth direction of the largest lumen region among the lumen regions extracted from the n/2 frames in each frame. Subsequently, the controller 130 extracts, from among the tracked lumen regions in the respective frames, the frame including the lumen region having the shallowest display depth (step S412).

The controller 130 determines whether the simple angle display mode is set to ON (step S414). When determining that the simple angle display mode is set to ON, the controller 130 proceeds to step S416. On the other hand, when determining that the setting of the simple angle display mode is OFF, the controller 130 proceeds to step S420.

The controller 130 performs the color tone conversion on the lumen region in the frame based on the color tone information allocated to the frame extracted in step S412 (step S416). First, the controller 130 performs processing related to the frame including the lumen region B1 of m=1.

The controller 130 performs the color tone conversion on the angle information allocated to the frame based on the color tone information allocated to the frame and displays the angle information in the information display region 204 of the examination screen 200 (step S418).

As shown in FIG. 20, when the processing related to the lumen region B1 of m=1 is completed, the controller 130 increments the variable m and sets m=2 (step S420).

The controller 130 tracks, in each frame, a change in the m-th largest lumen region in the depth direction among the lumen regions extracted from the n/2 frames. Subsequently, the controller 130 extracts the frame including the lumen region having the shallowest display depth from among the tracked lumen regions of the respective frames, and then cuts out the image information IFm in the lumen region (step S422). Since m=2 is set in step S420, the controller 130 performs processing related to the frame including the lumen region B2.

The controller 130 determines whether the simple angle display mode is set to ON (step S424). When determining that the simple angle display mode is set to ON, the controller 130 proceeds to step S426. On the other hand, when determining that the setting of the simple angle display mode is OFF, the controller 130 proceeds to step S430.

The controller 130 performs color tone conversion on the image information IFm based on the color tone information allocated to the frame of the image information IFm cut out in step S422 (step S426).

The controller 130 performs the color tone conversion on the angle information allocated to the frame based on the color tone information allocated to the frame and displays the angle information in the information display region 204 of the examination screen 200 (step S428).

The controller 130 synthesizes the image information IFm cut out from another frame with the m-th largest lumen region in the frame set as the base frame (step S430).

The controller 130 determines whether m=s has been established (step S432). When the variable m has become s, the controller 130 proceeds to step S434. On the other hand, when determining that m=s is not established, the controller 130 proceeds to step S436. The controller 130 increments the variable m (m=m+1) and returns to step S420.

The controller 130 causes the display part 120 to display, on the examination screen 200, the ultrasound image of the frame set as the base frame (step S434). Specifically, as illustrated in FIG. 9 and the like, the controller 130 pastes and synthesizes, in the lumen region of the frame as the base, the image information IFm cut out from within the lumen region of another frame.

On the other hand, returning to step S408 shown in FIG. 19, when the controller 130 determines that the lumen region is not extracted in the n frames of the ultrasound image, the process proceeds to step S444. In this case, all the acquired ultrasound images of the n frames have the brightness equal to or higher than the certain level, and are appropriate ultrasound images.

The controller 130 extracts the frame having the highest average brightness of the entire region among the n frames (step S444). After extracting the frame having high average brightness, the controller 130 proceeds to step S446.

The controller 130 determines whether the simple angle display mode is set to ON (step S446). When determining that the simple angle display mode is set to ON, the controller 130 proceeds to step S448. On the other hand, when determining that the setting of the simple angle display mode is turned off, the controller 130 proceeds to step S434 illustrated in step S20.

The controller 130 displays the angle information allocated to the frame extracted in step S444 in the information display region 204 of the examination screen 200 (step S448). After causing the angle information to be displayed, the controller 130 proceeds to step S434. The controller 130 causes the image display region 202 of the examination screen 200 to display the ultrasound image of the frame extracted in step S444 (step S434). Accordingly, the ultrasound image of the frame including the lumen region B having the highest brightness and the angle information of the ultrasound probe 150 when the ultrasound image is acquired are simultaneously displayed on the examination screen 200.

According to the third embodiment, substantially the same effects as those of the first embodiment described above can be achieved. That is, according to the third embodiment, the controller 130 extracts, in the predetermined frames, the continuous lumen region in which the brightness difference is within the threshold value TB, and tracks, in each frame, the change in the extracted lumen region in the depth direction. Subsequently, the controller 130 extracts the frame including the lumen region having the shallowest display depth from among the tracked lumen regions of the respective frames. That is, by performing the tilt-shift operation, the frame in a case where the ultrasound probe 150 and the lumen region are most orthogonal to each other can be automatically generated. Thus, an optimal ultrasound image can be acquired while performing the tilt-shift operation of the ultrasound probe 150 without using the accessory device. As a result, the optimal ultrasound image can be acquired by a simple operation without performing a special operation, and an increase in cost can also be prevented because no accessory device is required. In addition, the operator can acquire the optimum ultrasound image only by performing the tilt-shift operation of the ultrasound probe 150 without being conscious of the anisotropic reflection of the lumen structure.

Although the preferred embodiment of the present disclosure has been described in detail with reference to the accompanying drawings, the technical scope of the present disclosure is not limited to such examples. Further, those to which various modification examples and improvements have been applied naturally belong to the technical scope of the present disclosure within the category of the technical idea described in the scope of the claims of those skilled in the art.

Although embodiments of the present invention have been described and shown in detail, the disclosed embodiments are made for purposes of illustration and example only and not limitation. The scope of the present invention should be interpreted by terms of the appended claims.

The entire disclosure of Japanese Patent Application No. 2024-087780, filed on May 30, 2024, including description, claims, drawings and abstract is incorporated herein by reference.

What is claimed is:

1. An ultrasonic diagnostic apparatus that transmits an ultrasound wave into a subject, that receives the ultrasound wave reflected off a target tissue in the subject to obtain a reception signal, and that outputs an ultrasound image of the target tissue based on the reception signal, the ultrasonic diagnostic apparatus comprising:
a hardware processor,
wherein the hardware processor,
acquires a plurality of frames of the ultrasound image when an angle of an ultrasound probe is changed by a tilt-shift operation of the ultrasound probe,
extracts a standard frame including the ultrasound image in a case where the ultrasound probe and the target tissue are orthogonal to each other from the plurality of acquired frames, and
outputs the extracted standard frame.

2. The ultrasonic diagnostic apparatus according to claim 1, wherein the hardware processor extracts an image region in which a brightness correlation value between image regions included in each of the plurality of frames is equal to or smaller than a threshold value.

3. The ultrasonic diagnostic apparatus according to claim 2, wherein the hardware processor extracts, as the standard frame, a frame including the image region that has a highest average brightness from the extracted image region.

4. The ultrasonic diagnostic apparatus according to claim 1, wherein,
in a case in which each of the plurality of frames includes at least a first image region and a second image region,
the hardware processor extracts, from the plurality of frames, a first frame including the first image region having a highest average brightness among the first image regions as the standard frame, and
the hardware processor extracts, from the plurality of frames, a second frame including the second image region having the highest average brightness among the second image regions, cuts out the second image region from the second frame, and synthesizes the cut out second image region with the first image region of the first frame.

5. The ultrasonic diagnostic apparatus according to claim 1, wherein the hardware processor extracts a continuous lumen region in predetermined frames in which a brightness difference is within a threshold value among the plurality of frames, tracks a depth of the extracted lumen region in the plurality of frames, and extracts the frame including the lumen region having a shallowest depth as the standard frame from the plurality of frames.

6. An ultrasonic diagnostic apparatus that transmits an ultrasound wave into a subject, that receives the ultrasound wave reflected off a target tissue in the subject to obtain a reception signal, and that is capable of outputting an ultrasound image of the target tissue based on the reception signal, the ultrasonic diagnostic apparatus comprising:
a hardware processor,
wherein the hardware processor,
acquires a plurality of frames of the ultrasound image when an angle of an ultrasound probe is changed by a tilt-shift operation of the ultrasound probe, and
allocates angle information of the ultrasound probe with respect to a skin surface of a subject person when the tilt-shift operation is performed to each of the plurality of frames.

7. The ultrasonic diagnostic apparatus according to claim 6, wherein the hardware processor sets color tone information for each of the angle information allocated to the plurality of frames.

8. A non-transitory computer-readable storage medium storing a program executed in a computer of an ultrasonic diagnostic apparatus that transmits an ultrasound wave into a subject, that receives the ultrasound wave reflected off a target tissue in the subject to obtain a reception signal, and that is capable of outputting an ultrasound image of the target tissue on the basis of the reception signal, the program allowing the computer to perform:
acquiring a plurality of frames of the ultrasound image when an angle of an ultrasound probe is changed by a tilt-shift operation of the ultrasound probe,
extracting a standard frame including the ultrasound image in a case where the ultrasound probe and the target tissue are orthogonal to each other from the plurality of acquired frames, and
outputting the extracted standard frame.
